(19) United States
(12) Patent Application Publication
(10) Pub. No.: US 2007/0099907 A1
(43) Pub. Date: May 3, 2007

(54) Use of meloxicam in combination with an antiplatelet agent for treatment of acute coronary syndrome and related conditions

(76) Inventor: Raul Altman, Buenos Aires (AR)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/611,384

(22) Filed: Dec. 15, 2006

Related U.S. Application Data
(63) Continuation of application No. 11/275,829, filed on Jan. 31, 2006, now abandoned.
Application No. 11/275,829 is a continuation of application No. 11/094,384, filed on Mar. 29, 2005, now abandoned.
Application No. 11/094,384 is a continuation of application No. 10/315,806, filed on Dec. 10, 2002, now abandoned.

(51) Int. Cl. A61K 31/54

(52) U.S. Cl. 514/226.5

(57) ABSTRACT

The invention relates to a method of treatment or prevention of acute coronary syndrome or related conditions or reducing the risk of cardiovascular events comprising the administration of a therapeutically or prophylactically effective amount of meloxicam in combination with a therapeutically or prophylactically effective amount of an antiplatelet agent to a patient in need of such treatment. The invention also provides pharmaceutical compositions comprising meloxicam and an antiplatelet agent as a combined preparation suitable for use in these indications. Furthermore, the invention provides the use of meloxicam for manufacture of a pharmaceutical composition for treatment or prevention of acute coronary syndrome and related conditions when used in combination with an antiplatelet agent.

### RELATED APPLICATIONS

This is a continuation of application Ser. No. 11/275,829, filed Jan. 31, 2006, which is a continuation of Ser. No. 11/094,384, filed Mar. 29, 2005, now abandoned, which is a continuation of application Ser. No. 10/315,806, filed Dec. 10, 2002, now abandoned.

### TECHNICAL FIELD OF THE INVENTION

This invention relates to a method of treatment or prevention of acute coronary syndrome (ACS) and related conditions or reducing the risk of cardiovascular events, e.g. recurrent angina, percutaneous coronary revascularization, bypass grafting and cardiovascular mortality, comprising co-administration of effective amounts of the COX-2 selective NSAID meloxicam and an antiplatelet agent to a person in need of such treatment, suitable pharmaceutical compositions comprising meloxicam and an antiplatelet agent as a combined preparation for simultaneous, separate or sequential use in said method and the use of meloxicam for manufacture of a pharmaceutical composition for treatment or prevention of ACS and related conditions or for reducing the risk of cardiovascular events when used in combination with an antiplatelet agent.

### BACKGROUND OF THE INVENTION

Meloxicam (4-hydroxy-2-methyl-N-(5-methyl-2-thiazolyl)-2H-1,2-benzothiazine-3-carboxamide-1,1-dioxide) belongs to the NSAIDs (non-steroid-anti-inflammatory drugs) and is described in EP-A-0 002 482.

Orally administered, solid pharmaceutical preparations of meloxicam from which the active substance is released and absorbed rapidly, have been disclosed previously (WO 99/49867), as well as orally administered syrup formulations (WO 99/49845), and highly concentrated stable solutions (WO 01/97813).

Regarding the mechanism of action, meloxicam has been found to be a cyclooxygenase-2 (COX-2) and oxidoreductase inhibitor.

However, it has not been disclosed or suggested to use meloxicam for treatment and prevention of ACS.

NSAIDs (non steroidal anti-inflammatory drugs) are a class of compounds widely used for the treatment of inflammation, e.g. rheumatoid arthritis, pain and fever. It is well recognized that acetylsalicylic acid (ASA) and other NSAIDs exert their pharmacological effects through the non-selective inhibition of cyclooxygenase (COX) enzymes, thereby blocking prostaglandin synthesis (Nature, 231: 232-235, 1971). There are two types of COX enzymes, namely COX-1 and COX-2. COX-1 is expressed constitutively in many tissues, including the stomach, kidney and platelets, whereas COX-2 is expressed only at the site of inflammation (Gastroenterol., 111: 445-454, 1996). The prostaglandins derived from COX-1 are responsible for many of the physiological effects, including maintenance of gastric mucosal integrity. Reduction or avoidance of gastrointestinal toxicity was the motivation to develop COX-2-selective or even specific NSAIDs.

ASA (acetylsalicylic acid or aspirin), available for more than 100 years, because of its antiplatelet effects is still the drug of reference within the group of COX inhibitors for the treatment of acute coronary syndromes (ACS) and its effectiveness is well documented (Br. Med. J. 1994, 308: 81-106). It was shown that ASA is effective in patients with unstable angina or non-Q-wave acute myocardial infarction (MI), that it significantly reduces the incidence of MI, the mortality from cardiovascular causes and the incidence of non-fatal reinfarction. The combined incidence of cardiovascular death, MI and stroke in patients who had an ACS was shown to be reduced by 25% (N. Engl. J. Med. 1983, 309: 396-403; Lancet 1990: 827-30; N. Engl. J. Med. 1985, 313: 1369-1375; N. Engl. J. Med. 1988, 319: 1105-1111; Lancet 1988: 349-360).

Recently it has been reported that COX-2 inhibitors could increase cardiovascular events that this requires more investigation (Seminars in Urologic Oncology, 2001 Nov., 19(4), 294-305). Studies with first generation COX-2 inhibitors reveal that arthritic patients treated with rofecoxib have a five-fold higher risk of heart attack, compared to patients treated with naproxen (N. Engl. J. Med. 2000; 284: 1247-1255). Thus, like ASA, the non selective NSAID naproxen seems to exert cardioprotective effects, in contrast to selective COX-2 inhibitors such as rofecoxib.

The irreversible COX-1 inhibitor triflusal which shows very little inhibitory activity against endothelial COX-2 expression has been reported to be a valid alternative to ASA in treatment of unstable angina and for acute MI (European Heart Journal Supplements (2001) 3 (Supplement I), 123-130).

Furthermore, it has been reported that ASA, but not the more selective COX-2 inhibitors meloxicam and SC 58125, aggravates postischemic cardiac dysfunction (Naunyn-Schmiedebergs Archives of Pharmacology 363(2), 233-240 (2001)). It has also been reported that inhibition of COX-2 exacerbates ischemia-induced acute dysfunction in the rabbit (British Journal of Pharmacology 135(6), 1540-1546 (2002)).

WO 99/45913 discloses a combination therapy and preventive method for acute coronary syndrome and related conditions comprising administering to the patient a therapeutically effective amount of an antiplatelet agent in combination with a therapeutically effective amount of a COX-2 selective inhibitor. The antiplatelet agent may be a glycoprotein IIb/IIIa receptor antagonists, clopidogrel, ticlopidine, dipyridamole or ASA, the glycoprotein IIb/IIIa receptor antagonists being preferred. Furthermore, a series of specific COX-2 selective inhibitors is disclosed. The method is also disclosed to be useful in combination with other anticoagulants (e.g. thrombin inhibitors such as heparin, factor Xa inhibitors such as warfarin), and thrombolytic agents (e.g. streptokinase or tissue plasminogen activator). The indication is specified to include acute coronary ischemic syndrome, especially angina pectoris or first and subsequent Q-wave myocardial infarction, as well as thrombosis, thromboembolism, thrombotic occlusion and reocclusion, restenosis, transient ischemic attack and first or subsequent stroke.

It has now been found that a combination of the COX-2 selective NSAID meloxicam with an antiplatelet agent, optionally together with standard antithrombotic therapy such as heparin therapy provides an additional benefit regarding the risk of cardiovascular events as well as an cardioprotective effect, acting through the antiinflammatory effect of meloxicam. For instance, combining therapy of meloxicam with aspirin and heparin in patients with acute coronary syndromes without ST-segment elevation was found to decrease the triple end points of recurrent angina, myocardial infarction and vascular death or myocardial infarction, death and coronary revascularization procedures by 60%, indicating that is especially suitable for treatment of ACS patients.

### BRIEF SUMMARY OF THE INVENTION

There is a clear need for further alternative treatment regimes and improvement in the treatment and prevention of ACS (with/without elevation of the ST segment) in order to optimize treatment for a given patient, as well as for reducing the risk of cardiovascular events in patients at risk.

It is therefore an object of the invention to provide a method for treating and prevention of ACS (with/without elevation of the ST segment) and related conditions and for reducing the risk of cardiovascular events in patients at risk, comprising administering to a patient in need of such treatment an effective amount of meloxicam and an effective amount of an antiplatelet agent.

A second object of the invention is to provide pharmaceutical compositions as a combined preparation or a kit of parts comprising meloxicam and an antiplatelet agent for simultaneous, separate or sequential use in treatment or prevention of ACS and related conditions or for reducing the risk of cardiovascular events in patients at risk.

A third object of the invention is the use of meloxicam for manufacture of a pharmaceutical compositions as a combined preparation or a kit of parts comprising meloxicam and an antiplatelet agent for simultaneous, separate or sequential use in treatment or prevention of ACS and related conditions or for reducing the risk of cardiovascular events in patients at risk.

### DETAILED DESCRIPTION OF THE INVENTION

Viewed from a first aspect the present invention provides a novel method of treating, preventing or reducing the risk of occurrence of ACS or related conditions or reducing the risk of cardiovascular events comprising the administration of a therapeutically or prophylactically effective amount of meloxicam in combination with a therapeutically or prophylactically effective amount of an antiplatelet agent to a mammal, and more particularly, to a human patient. Thus the treatment basically uses inhibition of platelet aggregation and inhibition of inflammation in the vessel walls of the patient in need of such treatment. Vessel wall inflammation is known to influence the pathogenesis of unstable angina pectoris and myocardial infarction since inflammation markers such as C-reactive protein is of prognostic value for primary acute coronary syndromes (N. Engl. J. Med. 2002; 347: 1557-1565).

The method according to the invention can be added to standard antithrombotic therapy, preferably in high risk patients suffering from acute conditions which need hospitalization, for instance standard heparin therapy comprising the administration of low-molecular-weight heparin or unfractionated heparin, or administration of pentasaccharides, ximelagatran, melagatran, hirudin, hirulog, argatroban, lepirudin or bivalirudin as well as to oral anticoagulants such as warfarin or acenocumarol or can be applied after thrombolytic therapy (e.g. streptokinase or tissue plasminogen activator and related compounds).

In patients not suffering from acute conditions but at risk to develop acute coronary syndrome (ACS), related conditions or cardiovascular events it is preferred to apply the method of the invention without the standard antithrombotic therapy mentioned hereinbefore. In patients with a history of myocardial infarction at risk to develop acute coronary syndrome (ACS), related conditions or cardiovascular events, the method of the invention can be applied in combination with or without oral anticoagulants (secondary prevention, post-hospital treatment).

The indication “ACS or related conditions” should be understood in a non-limiting manner to comprise acute coronary ischemic syndrome, including unstable angina, non-ST-Segment elevation myocardial infarction (UA/NSTEMI), ST-segment elevation myocardial infarction (STEMI), occlusion or reocclusion of peripheral arteries, cerebral thromboembolism of extracraneal origin, transient ischemic attack and first or subsequent stroke.

The expression “cardiovascular events” should be understood in a non-limiting manner to comprise recurrent angina, percutaneous coronary revascularization, bypass grafting, cardiovascular mortality, coronary thrombotic occlusion and reocclusion or restenosis after percutaneous coronary revascularization and coronary artery bypass grafting.

Preferred indications with respect to the method of the invention are acute coronary ischemic syndrome including unstable angina, non-ST-Segment elevation myocardial infarction (UA/NSTEMI) and ST-segment elevation myocardial infarction (STEMI), and reducing the risk of cardiovascular events selected from recurrent angina, percutaneous coronary revascularization, bypass grafting and cardiovascular mortality.

Within the method of the invention meloxicam and the antiplatelet agent may be administered simultaneously together or at separate staggered times, i.e., sequentially. The beneficial effect is preferably achieved when the target plasma level concentrations of each active drug are maintained at substantially the same time.

“Acute coronary ischemia” refers to local anemia due to mechanical obstruction, e.g. arterial narrowing, of the blood supply. The condition is referred to as myocardial ischemia and is characterized by inadequate circulation of blood to the myocardium, usually as a result of coronary arterie disease. Ischemia of the heart muscle is evidenced by a pain in the chest often radiating from the precordium to the left shoulder and down the arm (angina pectoris) and is caused by coronary disease. Ischemia also includes myocardial infarction, which results from occlusion of a coronary artery. As used herein, the term “myocardial infarction” is intended to include both unstable angina and non-ST-Segment elevation myocardial infarction (UA/NSTEMI) as well as ST-segment elevation myocardial infarction (STEMI)., unless otherwise noted.

“Cerebrovascular ischemic events” relate to reduced blood supply to the brain, and include, but are not limited to, first or subsequent thrombotic strokes, or transient ischemic attacks.

“Therapeutically effective amount” is meant to be the amount of a drug or a pharmaceutical agent that will elicit the biological or medical response of a tissue, a system, animal or human that is being sought by a veterinarian, medical doctor or other clinician.

“Prophylactically effective amount” is meant to be the amount of a drug or a pharmaceutical agent that will prevent or reduce the risk of occurrence of the biological or medical event that is sought to be prevented in a tissue, a system, animal or human that is being sought by a veterinarian, medical doctor or other clinician.

“Reducing the risk” of occurrence of ACS or related conditions refers to lowering the risk of occurrence in a patient at risk to developing the conditions. Patients at risk to developing the conditions include those having a history of heart disease, a genetic predisposition to developing the conditions, diabetes, hyperlipidemia, hypertension and smokers.

Oral dosages of meloxicam when used for the indicated effects will range between about 0.01 mg per kg of body weight per day (mg/kg/day) to about 1.0 mg/kg/day, preferably 0.05 to 0.75 mg/kg/day, most preferably 0.1 to 0.4 mg/kg/day. For instance, an oral dosage of 15 mg could be given once a day to an individual patient. Suitable tablets, capsules or suspension (syrup) formulations for oral use or suppositories contain between 2.5 mg and 30 mg, preferably between 5 mg and 20 mg, most preferably between 7.5 mg and 15 mg, for example, 2.5 mg, 5 mg, 7.5 mg, 10 mg, 15 mg, 20 mg or 30 mg of meloxicam. Any of the formulations of meloxicam available on the market may be used in the method according to the invention. Reference is made to Rote Liste® 2002, Editio Cantor Verlag Aulendorf, Germany. Oral administration may be in one or divided doses of two, three, four, five or six times daily. A single daily dose is preferred.

Meloxicam could also be given in a bolus iv dose of about 10 to 20 mg daily, preferably a bolus iv dose of about 15 mg daily.

For parenteral administration meloxicam could be given in a dosage of 0.1 to 0.4 mg/kg body weight, preferably 0.2 to 0.3 mg/kg body weight, during 24 hours as slow i.v. infusion.

Antiplatelet agents suitable for use in the present invention include glycoprotein (GP) IIb/IIIa receptor antagonists, clopidogrel, ticlopidine, dipyridamole, cilostazol and ASA.

Herein, with respect to all aspects of the invention, the term “antiplatelet agent” is intended to include all pharmaceutically acceptable salts, ester and solvate forms, including hydrates, of compounds which have platelet aggregation inhibitory activity as well as pro-drug forms. The compounds having one or more chiral centers may occur as racemates, racemic mixtures and as individual diastereomers or enantiomers with all such isomeric forms and mixtures thereof being included within the scope of this invention. Any crystalline form of antiplatelet agent forming polymorphs are intended to be included in the invention.

GP IIb/IIIa receptor antagonists inhibit the binding of fibrinogen to the IIb/IIIa platelet receptor site, thereby inhibiting platelet aggregation. Suitable GP IIb/IIIa receptor antagonists are disclosed in WO 99/45913, page 22, line 35 to page 27, line 5, being herewith incorporated by reference. Suitable GP IIb/IIIa receptor antagonists are selected from DMP 754, sibrafiban, xemlofiban, fradafiban and orbofiban.

Preferred antiplatelet agents suitable for use in the present invention are selected from clopidogrel, dipyridamole and ASA. Especially preferred is ASA. Any of the formulations of clopidogrel, dipyridamole, cilostazol and ASA available on the market may be used in the method according to the invention. Reference is made to Rote Liste® 2002, Editio Cantor Verlag Aulendorf, Germany, and to Physicians Desk Reference, 56^{th} edition, 2002.

Oral dosages of GP IIb/IIIa receptor antagonists when used for the indicated effects will range between about 0.001 mg per kg of body weight per day (mg/kg/day) to about 50 mg/kg/day and preferably 0.005-20 mg/kg/day, most preferably 0.005-10 mg/kg/day. Suitable oral tablets and capsules contain between 0.1 mg and 5 g, preferably between 0.5 mg and 2 g, most preferably between 0.5 mg and 1 mg, for example, 0.5 mg, 1 mg, 5 mg, 10 mg, 150 mg, 250 mg or 500 mg of GP IIb/IIIa receptor antagonists. Oral administration may be in one or divided doses of two, three or four times daily. A single daily dose is preferred.

Intravenously, the most preferred doses for GP IIb/IIIa receptor antagonists will range from about 0.5 μg to about 5 mg/kg/minute during a constant rate infusion, to achieve a plasma level concentration during the period of time of administration of between 0.1 ng/ml and 1 μg/ml.

Clopidogrel can be administered orally in a daily dosage of about 25 mg to 500 mg, preferably from 75 to 375 mg, and most preferably from 75 to 150 mg. For example, a formulation or dosage unit may contain 25 mg, 50 mg, 75 mg, 150 mg, 250 mg or 500 mg of clopidogrel. Oral administration may be in one or divided doses of two, three or four times daily. A single daily dose is preferred.

Ticlopidine can be administered orally in a daily dosage of about 50 mg to 1000 mg, preferably from 100 to 750 mg, and most preferably from 200 to 500 mg. For example, a formulation or dosage unit may contain 50 mg, 100 mg, 200 mg, 250 mg or 500 mg of ticlopidine. Oral administration may be in one or divided doses of two, three or four times daily. A single daily dose is preferred.

Cilostazol can be administered orally in a daily dosage of about 50 mg to 500 mg, preferably from 100 to 300 mg, and most preferably from 150 to 250 mg. For example, a formulation or dosage unit may contain 50 mg, 100 mg, 200 mg, 250 mg or 500 mg of cilostazol. Oral administration may be in one or divided doses of two, three or four times daily. A single daily dose is preferred.

Dipyridamole can be administered orally in a daily dosage of about 25 to 500 mg, preferably 75 to 375 mg, most preferred 75 to 150 mg. For long-term treatment it is of advantage to administer repeated doses such as a dose of 25 mg dipyridamole retard or any other instant release formulation three or four times a day. For example, a formulation or dosage unit may contain 25 mg, 50 mg, 75 mg, 150 mg, 250 mg or 500 mg of dipyridamole. Oral administration may be in one or divided doses of two, three or four times daily. A single daily dose is preferred.

For parenteral administration dipyridamole could be given in a dosage of 0.5 to 5 mg/kg body weight, preferably 1 to 3.5 mg/kg body weight, during 24 hours as slow i.v. infusion (not faster than 0.2 mg/min).

Oral dosage amounts of ASA for the indicated effects generally range from about 10 mg to about 325 mg per day. For example, a formulation or dosage unit may contain 10 mg, 20 mg, 50 mg, 75 mg, 80 mg, 100 mg, 150 mg, 250 mg or 325 mg of ASA.

Standard heparin therapy which may be combined with the method of treatment according to the invention comprises the administration of low-molecular-weight heparin (LMWH), unfractionated heparin (UFH), hirudin, hirulog, argatroban, melagatran, lepirudin or bivalirudin, for instance subcutaneous LMWH (e.g. nadroparin 87 IU/kg BID or enoxaparin 1 mg/kg BID) or iv UFH given as an initial bonus of 5000 IU followed by continuous infusion at 1000 IU per hour for 7 days. The activated partial-thromboplastin time (APTT) can be used to assess the degree of anticoagulation in patients receiving iv UFH. Patients can be tested every 12 hours on the first day and every 24 hours subsequently. The APTT should be maintained in the range of 45 to 87 seconds (normal value, 30±5 seconds).

Viewed from a second aspect the present invention provides a pharmaceutical composition comprising a therapeutically or prophylactically effective amount of meloxicam and a therapeutically or prophylactically effective amount of an antiplatelet agent for use in treating, preventing or reducing the risk of occurrence of ACS or related conditions or reducing the risk of cardiovascular events. The instant pharmaceutical combinations include a single pharmaceutical dosage formulation containing both meloxicam and the antiplatelet agent in a pharmaceutical dosage formulation for simultaneous use, as well as a combined preparation or a kit of parts comprising meloxicam in a pharmaceutical dosage formulation and the antiplatelet agent in a separate pharmaceutical dosage formulation for simultaneous, separate or sequential use.

For instance, the kit of parts embodiment may be an oral dosage formulation of meloxicam and an oral dosage formulation of ASA. The packaging for the kit could be designed and manufactured in a variety of ways. A preferred example is a blister package containing rows of a meloxicam tablet and an ASA tablet side by side on the same blister card, each of the two tablets in its own blister bubble, with calendar or similar type markings on the card that convey to the user that one “pair” of tablets is to be ingested per day.

For administration the active compounds may be formulated with one or more conventional inert carriers and/or diluents as known in the art, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fatty substances such as hard fat or suitable mixtures thereof in conventional galenic preparations such as plain or coated tablets, lozenges, hard or soft capsules, dispersible powders or granules, syrups or elixirs, injectable solutions, ampoules, aqueous or oily suspensions, emulsions, solutions or suppositories.

Any of the formulations of meloxicam available on the market may be used in the pharmaceutical composition according to the invention. Reference is made to Rote Liste® 2002, Editio Cantor Verlag Aulendorf, Germany. Suitable tablets, capsules or suspension (syrup) formulations for oral use or suppositories contain between 2.5 mg and 30 mg, preferably between 5 mg and 20 mg, most preferably between 7.5 mg and 15 mg, for example, 2.5 mg, 5 mg, 7.5 mg, 10 mg, 15 mg, 20 mg or 30 mg of meloxicam.

GP IIb/IIIa receptor antagonists as antiplatelet agents may be used in oral tablets or capsules containing between 0.1 mg and 5 g, preferably between 0.5 mg and 2 g, most preferably between 0.5 mg and 1 mg, for example, 0.5 mg, 1 mg, 5 mg, 10 mg, 150 mg, 250 mg or 500 mg of the active compound. Suitable tablet and intravenous formulations containing a GP IIb/IIIa receptor antagonist are disclosed in WO 99/45913 as Examples 1 and 2.

Any of the formulations of clopidogrel, dipyridamole, cilostazol and ASA available on the market may be used in the pharmaceutical composition according to the invention. Reference is made to Rote Liste® 2002, Editio Cantor Verlag Aulendorf, Germany, and to Physicians Desk Reference, 56^{th} edition, 2002.

Clopidogrel as antiplatelet agent may be used for instance in oral tablets or capsules containing 25 mg, 50 mg, 75 mg, 150 mg, 250 mg or 500 mg of the active compound.

Ticlopidine as antiplatelet agent may be used for instance in oral tablets or capsules containing 50 mg, 100 mg, 200 mg, 250 mg or 500 mg of the active compound.

Cilostazol as antiplatelet agent may be used for instance in oral tablets or capsules containing 50 mg, 100 mg, 200 mg, 250 mg or 500 mg of the active compound.

Dipyridamole as antiplatelet agent may be used for instance in oral tablets or capsules containing 25 mg, 50 mg, 75 mg, 150 mg, 250 mg or 500 mg of the active compound.

ASA as antiplatelet agent may be used for instance in oral tablets or capsules containing 10 mg, 20 mg, 50 mg, 75 mg, 80 mg, 100 mg, 150 mg, 250 mg or 325 mg of the active compound.

Viewed from a third aspect the present invention provides the use of meloxicam in combination with an antiplatelet agent for the manufacture of a pharmaceutical composition for treating ACS or related conditions or for preventing or reducing the risk of occurrence of ACS or related conditions or reducing the risk of cardiovascular events.

The antiplatelet agent and preferred embodiments thereof, the indications and preferred embodiments thereof as well as pharmaceutical compositions are mentioned hereinbefore under the first and second aspect of the invention. Most preferred with respect to all aspects of the invention is the combination of meloxicam with ASA.

Compositions and methods of the invention may be used to treat, prevent or reduce the risk of formation of thrombi and thromboemboli and therefore to treat, prevent or reduce the risk of thrombotic occlusions and reocclusions. They are useful in surgery on peripheral arteries (arterial grafts, carotid endaterectomy) and in cardiovascular surgery where manipulation of arteries and organs, and/or the interaction of platelets with arterial surfaces, leads to platelet aggregation and potential forming of thrombi and thromboemboli. For example, the combination therapy can be used for preventing or reducing the risk of occurrence of platelet thrombosis, thromboembolism and reocclusion after acute intervention such as atherectomy, angioplasty, coronary artery bypass procedures or cardiac valve replacement. The combination therapy can also be used for preventing or reducing the risk of occurrence of platelet thrombosis, thromboembolism and reocclusion during and after thrombolytic therapy. Since blood vessels can also sustain chronic damage by the pathophysiological processes of atherosclerosis, patients with atherosclerosis can also be treated with the instant combination therapy to prevent or reduce the risk of occlusive thrombus formation. The instant combination therapy can be used to treat, prevent or reduce the risk of intermittent claudication, which is a clinical manifestation of peripheral vessel disease.

The instant combination therapy can also be used to treat, prevent or reduce the risk of a first or subsequent myocardial infarction in a person at risk for such events as well as to prevent or reduce the risk of restenosis in persons at risk for restenosis. Additionally, the instant combination therapy can be used for treating, preventing or reducing the risk of occurrence of acute cerebrovascular ischemic events (e.g. a first or subsequent thrombotic stroke, or transient ischemic attack). In general, the instant combination therapy can be used whenever antiplatelet therapy, or inhibition of platelet aggregation, is needed.

### EXAMPLE 1

### Efficacy Assessment of Combined Meloxicam/ASA in Acute Coronary Syndromes (ACS) Without ST-Segment Elevation

Background: Despite the use of heparin, aspirin and other antiplatelet agents, ACS patients without ST-segment elevation remain at risk of cardiovascular thrombotic events. It has been tested whether the combination of meloxicam and ASA would be superior to ASA alone in ACS patients under standard antithrombotic heparin therapy.

Summary: In an open-label, randomized, prospective, single-blind pilot study, patients with acute coronary syndromes without ST-segment elevation were randomized to aspirin and heparin treatment (n=60) or aspirin, heparin, and meloxicam (n=60) during coronary care unit stay. Patients then received aspirin or aspirin plus meloxicam for 30 days. During the coronary care unit stay, the primary outcomes variable of recurrent angina, myocardial infarction, or death was significantly lower in the patients receiving meloxicam (15.0% versus 38.3%, P=0.007). The second composite variable (coronary revascularization procedures, myocardial infarction, and death) was also significantly lower in meloxicam-treated patients (10.0% versus 26.7%, P=0.034). At 90 days, the primary end point remained significantly lower in the meloxicam group (21.7% versus 48.3%, P=0.004), as did the secondary end point (13.3% versus 33.3%, P=0.015) and the need for revascularization alone (11.7% versus 30.0%, P=0.025). No adverse complications associated with the meloxicam treatment were observed.

Methods

An open, randomized, prospective, single-blind pilot study of the use of meloxicam in ACS patients without ST-segment elevation was performed. Patients were recruited from the Centro de Salud and the Centro Modelo de Cardiologia, Tucuman, Argentina. Inclusion criteria were chest pain within the previous 24 hours associated with ST-segment depression (>0.5 mm) and either ECG evidence of ischemia or previously documented coronary artery disease. Exclusion criteria were persistent ST-segment elevation and increased levels of the MB isoenzyme of creatine kinase (CK-MB) consistent with acute myocardial infarction, revascularization procedures within the preceding 6 months, malignancy, pregnancy, renal or hepatic diseases, use of anticoagulant therapy, treatment with anti-inflammatory drugs, or contraindication of the study drugs. Patients were randomized to receive aspirin plus heparin (n=60) or aspirin, heparin, and meloxicam (n=60). The active treatment group received 15 mg of meloxicam given intravenously immediately after randomization followed by 15 mg once daily given orally during hospital stay and for 30 days after discharge. Both groups received aspirin for 30 days. The dose range was 100 to 300 mg per day at the discretion of the treating physician. CK-MB levels were measured before randomization. Additional assays were performed after an episode of chest pain to confirm the diagnosis of acute MI. All patients received subcutaneous LMWH (either nadroparin 87 IU/kg BID or enoxaparin 1 mg/kg BID) or intravenous UFH given as an initial bolus of 5000 IU followed by continuous infusion at 1000 IU per hour for 7 days or until discharge from the hospital. The activated partial-thromboplastin time (APTT) was used to assess the degree of anticoagulation in patients receiving intravenous unfractionated heparin. Patients were tested every 12 hours on the first day and every 24 hours subsequently. The APTT was maintained in the range 45 to 87 seconds (normal value, 30±5 seconds).

Study End Points

The primary outcome variable was the composite of recurrent angina, MI, or death during CCU stay and after 90 days of follow-up. An additional secondary composite variable was determined: MI, death, and all revascularization procedures (percutaneous transluminal coronary angioplasty [PTCA] or coronary artery graft surgery [CABG]). Each component of the primary and secondary outcome variable was also recorded separately. According to Degner et al, Drugs Today 1998, 34 (suppl A):1-22, after bolus intravenous dose of 15 mg meloxicam, Cₘₐₓ was 3.0 mg/L and tₘₐₓ was 0.05 hours. These figures anticipated that intravenous administration of meloxicam would produce an immediate anti-inflammatory effect, and all events after randomization were considered as end points. Cardiac catheterization was performed at the discretion of treating physicians, but only urgent PTCA and CABG, usually after recurrent angina or MI, were considered as end points. Revascularization procedures were indicated after recurrence of chest pain refractory to medical treatment. During CCU stay and after discharge, all patients were examined for clinical events by an investigator who was unaware of treatment allocation. Patients were also instructed to report any events to study personnel immediately. The diagnostic criteria for MI were chest pain lasting 20 minutes or more with ECG changes and an at least 2-fold elevation of CK-MB. Recurrent angina was defined as recurrence of chest pain with ECG changes refractory to medical therapy (aspirin, heparin, nitroglycerin, and β-blockers).

Statistical Analysis

The nature of the quantitative variables distribution was assessed by the Shapiro-Wilk test. Differences between the 2 treatment groups were analyzed by the Student's t test (independent samples) or the Mann-Whitney test, according to the nature of the distribution. Differences in terms of activated partial thromboplastin time after unfractionated heparin were assessed by a 2-way ANOVA model, with repeated measures in one of the factors (Student Newman-Keuls post-hoc test). Univariate associations between qualitative variables were assessed by X² (Yates corrected) and Spearman rank-order correlations.

Relative risks (RRs) and their 95% CI were obtained. The RR reduction (RRR) is defined as 1 minus RR and expressed as percentage. The absolute risk reduction (ARR) was calculated as the difference between the risk in control patients minus the risk in treated patients and expressed as percentage. A multiple logistic regression model was used to determine the predictive value of the variables associated with events at day 90 in the previous univariate analysis. The software used were CSS/Statistics 3.1 (StatSoft Corp) and EPI INFO 6 v.6.04, 1996.

Results

In total, 120 patients were randomized. Sixty patients received meloxicam with aspirin and heparin, and a control group of 60 patients were given heparin and aspirin only. The baseline clinical characteristics of the patients in the 2 groups did not differ significantly (Table 1). There were more male patients in the control group (37 patients, 61.7%) than in the meloxicam group (31 patients, 51.7%), and the mean CCU stay was longer in the control group (4.4±1.6 versus 4.35±1.14 days), but these differences were not significant.

TABLE 1
Baseline Clinical Characteristics of Patients

| | Meloxicam | Control Group | |
|---|---|---|---|
| | (n = 60) | (n = 60) | P |
| Sex | | | |
| Male | 31 (51.7) | 37 (61.7) | 0.357 |
| Female | 29 (48.3) | 23 (38.3) | . . . |
| Age, y | | | |
| Mean | 61 | 60.7 | 1.0 |
| Range | 38-84 | 28-81 | . . . |
| Risk factors | | | |
| Smoking | | | |
| Current | 21 (35.0) | 22 (36.7) | 1.0 |
| Former* | 3 (5.0) | 8 (13.3) | 0.206 |
| Never | 36 (60.0) | 30 (50.0) | 0.359 |
| Body mass index | | | |
| ≦25 | 17 (28.3) | 10 (16.7) | 0.19 |
| >25 | 43 (71.7) | 46 (76.3) | . . . |
| Dislipidemia | 17 (28.3) 1 | 7 (28.3) | 1.0 |
| Diabetes | 13 (21.7) | 22 (36.7) | 0.108 |
| Hypertension | 22 (36.7) | 27 (45.0) | 0.458 |
| Previous myocardial infarction | 11 (18.3) | 11 (18.3) | 1.0 |
| Previous unstable angina | 2 (3.3) | 0 | . . . |
| Previous PTCA | 4 (6.6) | 8 (13.3) | 0.361 |
| Previous CABG | 3 (5.0) | 2 (3.3) | 1.0 |

Values are n (%) unless otherwise indicated.
*Stopped smoking >12 months ago.

TABLE 2
Activated Partial Thromboplastin Time After Unfractionated Heparin

| | Activated Partial Thromboplastin Time, s | | | | | | | |
|---|---|---|---|---|---|---|---|---|
| | Day 1 | | Day 2 | | Day 3 | | Day 4* | |
| | Control | Melox | Control | Melox | Control | Melox | Control | Melox |
| | (n = 36) | (n = 50) | (n = 36) | (n = 50) | (n = 35) | (n = 50) | (n = 28) | (n = 47) |
| Mean ± SD, s | 54.9 ± 22.4 | 55.8 ± 26.4 | 70.4 ± 23.0 | 69.6 ± 19.9 | 78.9 ± 22.2 | 89.9 ± 24.4 | 72.5 ± 18.5 | 89.3 ± 20.8 |
| Median, s | 48 | 48 | 63 | 62 | 77 | 86 | 74 | 84.5 |
| APTT in | 56.9 | 67.7 | 77.8 | 79.7 | 61.2 | 52.3 | 88.9 | 58.3 |
| therapeutic | | | | | | | | |
| range | | | | | | | | |
| (45-87 s), % | | | | | | | | |
| APTT | 33.3 | 25.8 | 4.4 | 3.6 | 2.7 | 0 | 0 | 0 |
| under | | | | | | | | |
| therapeutic | | | | | | | | |
| range | | | | | | | | |
| (<45 s), % | | | | | | | | |
| APTT | 9.8 | 6.5 | 17.8 | 16 | 36.1 | 47.7 | 11.1 | 41.7 |
| over | | | | | | | | |
| therapeutic | | | | | | | | |
| range | | | | | | | | |
| (>87 s), % | | | | | | | | |

Melox indicates meloxicam group. n is the number of patients treated with unfractionated heparin.
*Control and meloxicam group, P < 0.05 (ANOVA).

Ten patients in the meloxicam group and 24 patients in the control group were treated with LMWH. The remaining patients received UFH. In the meloxicam group, 10 patients were treated with nadroparin. In the control group, 22 patients received nadroparin and 2 received enoxaparin. The capacity of unfractionated heparin to maintain the APTT in the therapeutic range (45 to 87 seconds) is shown in Table 2. Within the first 24 hours, 56.9% of those receiving UFH in the control group and 67.7% in the meloxicam group had an APTT in the therapeutic range and 9.8% and 6.5%, respectively, had an APTT above the therapeutic range. Thirty three percent and 26% (P=0.618), respectively, in the control and meloxicam groups of patients had subtherapeutic APTT levels during the first 24 hours. After the first day, <5% of patients in either group were under the therapeutic range, and by day 4 all patients were within or above the therapeutic range.

Primary Outcome Variables

Efficacy Outcomes Within the CCU Period

The incidence of recurrent angina was significantly lower in the meloxicam-treated group compared with the control group (9 of 60 patients, 15.0% versus 21 of 60 patients, 35%; P=0.02). This corresponds to a RRR of 57.1% (95% CI, 14 to 79) (Table 3). There was a trend toward a reduced need for revascularization procedures in patients who received meloxicam, although this did not reach statistical significance (P=0.055). Fewer composite events (recurrent angina, MI, and death) occurred in the meloxicam group (9 patients; 15%) compared with the control group (23 patients; 38.3%). The difference was statistically significant (P=0.007) and corresponds to a RRR of 60.8%, (95% CI, 23 to 80). Composite outcomes for coronary revascularization procedures (PTCA or CABG), MI, and death occurred in 6 patients in the meloxicam group (10%) and 16 patients of the control group (26.7%). This difference was also statistically significant (P=0.034), with a RRR of 62.5% (95% CI, 11 to 84). Treatment was discontinued during the in-hospital period for 8 patients in the control group and 3 in the meloxicam group. The reasons for stopping were the occurrence of an end point in 5 patients in the control group and 2 patients in the meloxicam group. One patient in each group withdrew consent, and treatment was discontinued in 2 patients assigned to the control group because of discharge from hospital.

Efficacy Outcomes at 90 Days

The differences between the meloxicam-treated group and the control group observed during the period of hospitalization were maintained during the follow-up period. The cumulative frequency of recurrent angina during the 90-day follow-up period was lower in the meloxicam group (12 patients; 20%) compared with the control group (26 patients; 43.3%). The difference was statistically significant (P=0.011) and corresponded to a RRR of 53.8% (95% CI, 17 to 74) (Table 4). Significantly fewer patients in the meloxicam group required coronary revascularization (7 patients, 11.7% versus 18 patients, 30%; P=0.025, RRR 61%, 95% CI, 14 to 82). The cumulative composite end point of recurrent angina, MI, and death occurred in 13 patients (21.7%) in the meloxicam group and in 29 patients (48.3%) in the control group. The difference was statistically significant (P=0.004) and was associated with a RRR of 55.1% (95% CI, 22 to 74) for the meloxicam group. The cumulative frequency of the secondary outcome variable of coronary revascularization procedures, MI, and death was significantly lower in the meloxicam group (8 patients; 13.3%) compared with the control group (20 patients; 33.3%) (P=0.015) and corresponded to a RRR of 60.1% (95% CI, 16 to 81) (Table 4).

TABLE 3
Outcomes During In-Hospital Stay

| | Events During the Coronary Care Unit Period | | | | |
|---|---|---|---|---|---|
| | Meloxicam | Control | RRR | ARR | |
| | (n = 60) | (n = 60) | (IC 95%) | % | P* |
| Recurrent angina, | 9 (15.0) | 21 (35.0) | 57.1 (14-79) | 20.0 | 0.02 |
| n (%) | | | | | |
| All | 6 (10.0) | 15† | 60.1 | 15.0 | 0.055 |
| revascularization, | | (25.0) | (4.0-83) | | |
| n (%) | | | | | |
| PTCA, n | 2 | 4 | . . . | . . . | . . . |
| CABG, n | 4 | 10 | . . . | . . . | . . . |
| AMI, n | 0 | 2 | . . . | . . . | . . . |
| Vascular death, n | 0 | 1 | . . . | . . . | . . . |
| Recurrent | 9 (15.0) | 23 (38.3) | 60.8 (23-80) | 23.3 | 0.007 |
| angina + MI + | | | | | |
| vascular death, | | | | | |
| n (%) | | | | | |
| MI + death + | 6 (10.0) | 16 (26.7) | 62.5 (11-84) | 16.7 | 0.034 |
| revascularization, | | | | | |
| n (%) | | | | | |

*χ² test.
†One patient underwent both percutaneous revascularization and bypass grafting. Only 1 event per patient was considered.

TABLE 4
Outcomes During 90-Day Follow-Up

| | Events During 90-Day Follow-up | | | | |
|---|---|---|---|---|---|
| | Meloxicam | Control | RRR | | |
| | (n = 60) | (n = 60) | (95% CI) | ARR % | P* |
| Recurrent angina, n (%) | 12 (20.0) | 26 (43.3) | 53.8 (17-74) | 23.3 | 0.011 |
| Revascularization, n (%) | 7 (11.7) | 18† (30.0) | 61.0 (14-82) | 18.3 | 0.025 |
| PTCA, n | 3 | 7 | . . . | . . . | . . . |
| CABG, n | 4 | 12 | . . . | . . . | . . . |
| MI, n (%) | 0 | 3 (5) | . . . | . . . | . . . |
| Vascular death, n (%) | 0 | 2 (3.3) | . . . | . . . | . . . |
| Recurrent | 13 (21.7) | 29 (48.3) | 55.1 (22-74) | 26.6 | 0.004 |
| angina + MI + vascular death, n | | | | | |
| (%) | | | | | |
| MI + death + revascularization, | 8 (13.3) | 20 (33.3) | 60.1 (16-81) | 20.0 | 0.015 |
| n (%) | | | | | |

*χ² test.
†One patient underwent both percutaneous revascularization and bypass grafting. Only 1 event per patient was considered.

TABLE 5
Multivariate Association Between Events and Treatment Group

| Variable | Odds Ratio | 95% CI | P |
|---|---|---|---|
| Hypertension | 2.31 | 1.00-5.38 | <0.05 |
| Diabetes | 2.43 | 1.01-6.36 | <0.05 |
| Treatment Group | 0.33 | 0.14-0.77 | 0.01 |

Logistic regression (maximum likelihood). χ² = 22.36; df: 4, P = 0.0002.

Temporal Trends

Within 48 hours of randomization, the primary outcome variable had been reached by 1.7% of patients in the meloxicam group and 6.7% of patients in the control group (P=0.361). At 30 days, the rates were significantly lower in the meloxicam-treated patients (20.0% versus 46.7%; P=0.004). The additional secondary outcome variable was reached at 30 days in 11.7% of the meloxicam group compared with 31.7% of the control group (P=0.015). The univariate Spearman rank-order correlations showed a significant association between events during the 90-day follow-up period and hypertension (rS=0.24, P=0.007), hypercholesterolemia (rS=0.20, P=0.024), diabetes mellitus (rS=0.26, P=0.004), and treatment group (rS=−0.28, P=0.002). In the multivariate analysis (Table 5), only hypertension, diabetes, and treatment group were significantly associated with events during the 90-day follow-up period.

Safety

No bleeding complications, side effects, or intolerance was observed in any patients throughout the course of the study. This agrees with the fact that in general pharmacologic evaluations with dosing regimens 3- to 10-fold higher than the anti-inflammatory doses of meloxicam, no evidence of impairment of the central nervous, cardiovascular/pulmonary, renal, musculoskeletal, or autonomic systems was observed (Degner et al, Drugs Today 1998, 34 (suppl A): 1-22).

Discussion

An initial significant reduction in the number of events was observed during the CCU period, which was sustained through the 90-day follow-up period. The significant difference between the control and meloxicam groups was driven by the differences in the rates of recurrent chest pain, with a RRR of 57.1%, an absolute risk reduction (ARR) of 20%, and a reduction in the need for myocardial revascularization procedures (RRR 60.8%; ARR 15%) during the CCU period. The ARR in recurrent angina improved during the 30-day treatment period by 3.3%. At 90 days of follow-up, the differences were sustained with only one additional death reported in each group.

1. A method for the treatment or prevention of acute coronary syndrome or a related condition or reducing the risk of a cardiovascular event comprising the administration of a therapeutically or prophylactically effective amount of meloxicam in combination with a therapeutically or prophylactically effective amount of an antiplatelet agent to a patient in need of such treatment.

2. The method of claim 1, added to standard antithrombotic therapy or applied after thrombolytic therapy.

3. The method of claim 1, wherein the patient is a high risk patient suffering from acute acute coronary syndrome or related conditions which need hospitalization or is a patient at risk to develop coronary syndrome (ACS), related conditions or cardiovascular events.

4. The method of claim 1 wherein the acute coronary syndrome or related condition is selected from the group consisting of acute coronary ischemic syndrome, including unstable angina, non-ST-segment elevation myocardial infarction (UA/NSTEMI), ST-segment elevation myocardial infarction (STEMI), occlusion or reocclusion of peripheral arteries, cerebral thromboembolism of extracraneal origin, transient ischemic attack and first or subsequent stroke, and wherein the cardiovascular event is selected from the group consisting of recurrent angina, percutaneous coronary revascularization, bypass grafting, cardiovascular mortality, coronary thrombotic occlusion and reocclusion or restenosis after percutaneous coronary revascularization and coronary artery bypass grafting.

5. The method of claim 1 wherein the acute coronary syndrome or related condition is selected from the group consisting of acute coronary ischemic syndrome including unstable angina, non-ST-Segment elevation myocardial infarction (UA/NSTEMI) and ST-segment elevation myocardial infarction (STEMI), and wherein the cardiovascular event is selected from the group consisting of recurrent angina, percutaneous coronary revascularization, bypass grafting and cardiovascular mortality.

6. The method of claim 1 wherein the antiplatelet agent is selected from the group consisting of glycoprotein (GP) IIb/IIIa receptor antagonists, clopidogrel, ticlopidine, dipyridamole, cilostazol and acetylsalicylic acid.

7. The method of any of claim 1 wherein the antiplatelet agent is selected from the group consisting of clopidogrel, ticlopidine, dipyridamole, cilostazol and acetylsalicylic acid.

8. The method of any of claim 1 wherein the antiplatelet agent is selected from the group consisting of ticlopidine, dipyridamole and acetylsalicylic acid.

9. A pharmaceutical composition comprising a therapeutically or prophylactically effective amount of meloxicam and a therapeutically or prophylactically effective amount of an antiplatelet agent.

10. The composition of claim 9 as a combined preparation or a kit of parts comprising meloxicam in a pharmaceutical dosage formulation and the antiplatelet agent in a separate pharmaceutical dosage formulation for simultaneous, separate or sequential use.
